(19) United States
(12) Patent Application Publication
(10) Pub. No.: US 2026/0102623 A1
(43) Pub. Date: Apr. 16, 2026

(54) HEADER ASSEMBLY FOR AN IMPLANTABLE INTRACARDIAC DEVICE AND RESPECTIVE INTRACARDIAC DEVICE

(71) Applicant: BIOTRONIK SE & Co. KG, Berlin (DE)

(72) Inventors: Martin HENSCHEL, Berlin (DE); Ville POHJAMO, Berlin (DE); Peter LIEBISCH, Berlin (DE); Ringo MANNHAUPT, Luckenwalde (DE); Marco BOSSELMANN, Rostock (DE); Ruien WALI, Berlin (DE); Jens RUMP, Berlin (DE); Ingmar STADE, Berlin (DE); Martin HESSING, Berlin (DE); Torsten HOMUTH, Ostseebad Nienhagen (DE)

(73) Assignee: BIOTRONIK SE & Co. KG, Berlin (DE)

(21) Appl. No.: 19/116,717

(22) PCT Filed: Sep. 19, 2023
(86) PCT No.: PCT/EP2023/075727

§ 371 Date: Mar. 28, 2025

(30) Foreign Application Priority Data
Oct. 7, 2022 (EP) 22200200.8

(51) Int. Cl. A61N 1/375

(52) U.S. Cl. CPC A61N 1/3754; A61N 1/37512; A61N 1/3756

(57) ABSTRACT

Header assembly for an implantable intracardiac device which is automated assembly friendly, wherein the header assembly comprises a cylindrical feedthrough arrangement having an outer shell surface, a ring-shaped proximal cap including an inner surface, a ring-shaped distal cap including an outer surface, a base ring with at least two tines protruding distally from the base ring, and a pressing arrangement. The distal cap includes a fixing portion with an inner surface forming a locking connection with the feedthrough arrangement outer shell surface for counteracting a movement of the distal cap and the feedthrough arrangement apart from each other in an axial direction. The proximal cap inner surface and the distal cap outer surface are arranged coaxially and directed towards each other, and the base ring is interposed between the proximal cap inner surface and the distal cap outer surface such as to be coaxially rotatable relative to the distal cap.

### CROSS-REFERENCE TO RELATED APPLICATIONS

This application is the United States National Phase under 35 U.S.C. § 371 of PCT International Patent Application No. PCT/EP2023/075727, filed on Sep. 19, 2023, which claims the benefit of European Patent Application No. 22200200.8, filed on Oct. 7, 2022, the disclosures of which are hereby incorporated by reference herein in their entireties.

### TECHNICAL FIELD

The present invention refers to an implantable intracardiac device, such as an implantable intracardiac pacemaker, and a header assembly therefor as well as a manufacturing method for such header assembly and for such implantable intracardiac device.

### BACKGROUND

Active or passive medical devices such as implantable intracardiac devices, for example implantable intracardiac pacemakers (also known as leadless pacemakers), are well known miniaturized medical devices which are entirely implanted into a heart's ventricle or atrium. Intracardiac pacemakers are used for patients who suffer from a bradycardia, that is if a heart beats too slow to fulfil the physiological needs of the patient. Intracardiac pacemakers apply electrical stimulation in the form of pulses to the heart in order to generate a physiologically appropriate heartrate and/or in the form of shocks for cardioversion or defibrillation in order to restore a more normal heart rhythm. Alternative or additional functions of intracardiac devices comprise providing other electrical or electromagnetic signals to the heart or its surrounding tissue, sensing electrical or electromagnetic signals or other physiological parameters of the heart and/or its surrounding tissue.

Documents US 2012/0172690 A1 and U.S. Pat. No. 10,112,045 B2 disclose a leadless pacemaker device which comprises a conductive housing, and a fixation element assembly. The fixation element assembly includes a set of active fixation tines and an insulator to electrically isolate the set of active fixation tines from the conductive housing of the implantable medical device. The active fixation tines in the set are deployable from a spring-loaded position in which distal ends of the active fixation tines point away from the implantable medical device to a hooked position in which the active fixation tines bend back towards the implantable medical device. The active fixation tines are configured to secure the implantable medical device to a patient tissue when deployed while the distal ends of the active fixation tines are positioned adjacent to the patient tissue.

However, known manufacturing methods for leadless pacemakers need sophisticated alignment methods to fix a tine array to a medical implant housing. For example: Exact orienting of miniature components to each other is needed before assembly or the sophisticated dispensing of adhesive material in microgram dosages or an alignment of delicate bayonet features is needed to combine a header with a housing. Furthermore, complicated injection molded parts with notches are needed for the fixation of the tines. Such manufacturing steps are hardly suitable for automatization, also since silicone adhesive manual cleaning procedures are needed after assembly.

Additionally, known headers take space from other critical components of the implantable intracardiac device, such as the battery or electronics module. Accordingly, smaller header size is desirable.

Furthermore, upon implantation of the intracardiac device, it may be desired to be able to re-orient portions of the intracardiac device relative to its header assembly or particularly relative to the tines being fixed in heart tissue. Such re-orienting process may be required e.g. for establishing a desired communication orientation upon using coil induced electrical field communication. However, during operation of the intracardiac device, i.e. after having completed the implantation procedure, any unintended change in the orientation of the intracardiac device should be prevented.

Accordingly, there may be a need for implantable intracardiac devices and respective header assemblies and for methods for manufacturing same addressing at least one of the above-mentioned requirements. Particularly, there may be a need for a header assembly having small dimensions, providing a reliable mechanism for fixing the intracardiac device at heart tissue, enabling setting an orientation of the cardiac device during implantation and maintaining the orientation during subsequent device operation and/or enabling low manufacturing effort and costs.

Such need may be fulfilled by the subject matter of one of the independent claims. Advantageous embodiments are defined in the dependent claims, described in the present specification and visualised in the associated figures.

The present disclosure is directed toward overcoming one or more of the above-mentioned problems, though not necessarily limited to embodiments that do.

### SUMMARY

According to a first aspect of the present invention, a header assembly for an implantable intracardiac device is described. The header assembly comprises a cylindrical feedthrough arrangement, a ring-shaped proximal cap, a ring-shaped distal cap, a base ring with at least two tines protruding distally from the base ring, and a pressing arrangement. The feedthrough arrangement has an outer shell surface. The proximal cap comprises an inner surface. The distal cap comprises an outer surface. The distal cap comprises a fixing portion with an inner surface. The inner surface forms a locking connection with the outer shell surface of the feedthrough arrangement for counteracting a movement of the distal cap and the feedthrough arrangement apart from each other in an axial direction. The proximal cap, the distal cap and the base ring are configured such that the inner surface of the proximal cap and the outer surface of the distal cap are arranged coaxially and directed towards each other, and the base ring is interposed between the inner surface of the proximal cap and the outer surface of the distal cap such as to be coaxially rotatable relative to the distal cap. The pressing arrangement is configured such as to exert an elastic force in a radial direction such as to press the base ring against one of the inner surface of the proximal cap and the outer surface of the distal cap.

Only as some introductory or summarising notes and without limiting the scope of the invention, basic ideas underlying embodiments of the invention and associated possible advantages may be roughly described as follows:

The header assembly presented herein is specifically configured for enabling a simple but nevertheless reliable assembling procedure upon mounting the header assembly to a housing of an implantable intracardiac device (hereinafter: “ID”). Particularly, the ring-shaped proximal and distal caps may be easily pressed in an axial direction onto the cylindrical feedthrough arrangement provided at a distal end of the housing of the ID. Therein, the outer shell surface of the feedthrough arrangement and the inner surface of the fixing portion of the distal cap are specifically configured such that, upon being actually pressed together, a preferably non-reversible, i.e. permanent, locking connection such as a snap-fit connection or a press-fit connection is established between both components. Due to such locking connection, the header assembly is reliably held at the housing of the ID.

Furthermore, the base ring with its at least two tines is interposed between the inner surface of the proximal cap and the outer surface of the distal cap and is therefore also reliably held at the housing of the ID. Specifically, the base ring is arranged and configured such as to being coaxially rotatable relative to the distal cap. As the distal cap is fixed via the feedthrough arrangement to the housing of the ID, the base ring is therefore rotatable with respect to the housing. On the one hand, such rotation capability may be used during an implantation procedure to correctly orientate the housing with respect to the tines extending from the base ring, these tines being fixed to cardiac tissue in order to correctly hold the entire ID. However, on the other hand, it should be prevented that such initially correct orientation is subsequently modified during the normal operation of the ID (i.e. after completion of the implantation procedure) for example due to rotation forces acting onto the ID during normal heartbeat and/or during motions of the patient. Accordingly, on the one hand, the base ring with its tines should be prevented from rotating relative to the housing of the ID as long as only minor rotation forces act onto the housing, such minor rotation forces being lower than rotation forces typically being applied to the housing during normal operation. On the other hand, rotating the base ring relative to the housing of the ID should be enabled upon major rotation forces acting onto the housing, such major rotation forces being for example applied during an implantation procedure for specifically orienting the ID housing.

In order to establish such specific rotation capability, it was tested that the base ring is clamped by a clamping action between the proximal cap and the distal cap such that it may only be rotated upon rotation forces being applied such as to exceed friction forces between the base ring, on the one side, and the proximal and distal caps, on the other side, the friction forces resulting from the clamping action. However, it has been observed that, in order to establish such clamping action, the distal cap would generally have to be mounted with its fixing portion on the feedthrough arrangement in a configuration in which substantial permanent mechanical stress is applied to the distal cap and its fixing portion. Although such distal cap is preferably being made from a high quality polymeric material such as PEEK, it has been observed that such permanent mechanical stress may result in mechanical failures or damages of the material of the distal cap, such effects also being referred to as environmental stress cracking (ESC). Particularly, such ESC preferably occurs upon polymeric components of the header assembly being loaded under a certain amount of mechanical stress (static or cyclic) and are exposed to an oxidative environment, such as a contact with human blood. However, any ESC occurring at the fixing portion of the distal cap might result in failure of the locking connection between such fixing portion and the outer shell surface of the feedthrough arrangement. Upon such failure, in a worst-case, the distal cap could detach from the feedthrough arrangement thereby releasing the entire fixation of the ID housing to the base ring and the tines fixed to the cardiac tissue. Of course such releasing action shall be prevented.

In order to suppress such occurrence of ESC, the distal cap may be adapted such that its fixing portion may be pushed onto the feedthrough arrangement during an assembling procedure and may then form a locking connection in which the fixing portion is not permanently mechanically stressed beyond a degree at which ESC typically occurs. Furthermore, in order to ensure that a sufficient rotation friction is established between the proximal and distal caps and the base ring interposed between those, the header assembly furthermore comprises a specific pressing arrangement. Such pressing arrangement is configured such that an elastic force is exerted onto the base ring, thereby pressing the base ring against the proximal or the distal cap such as to finally establish the required rotation friction. Accordingly, correctly orienting the ID housing during an implantation procedure may be possible by applying rotation forces exceeding the rotation friction induced by the pressing arrangement whereas unintended miss-orienting of the ID housing during later operation is prevented as generally no rotation forces exceeding the rotation friction are induced during such normal operation of the ID. Furthermore, even in cases where excessive mechanical stress acts onto the pressing arrangement and therefore damages such as cracks eventually occur at the pressing arrangement for example due to ESC, such damages or cracks would only affect the pressing arrangement but not the rest of the header assembly and particularly not the fixing portion of the distal cap. Accordingly, even in case of such damages or cracks, an integrity of the entire header assembly and particularly a fixation of the distal to the feedthrough arrangement are not jeopardized.

Subsequently, possible features of embodiments of the invention and associated possible advantages will be described in more detail.

The implantable intracardiac device may be, for example, an implantable intracardiac pacemaker (also known as leadless pacemaker) which may apply electrical stimulation in the form of pulses to the heart in order to generate a physiologically appropriate heartrate and/or in the form of shocks for cardioversion or defibrillation in order to restore a more normal heart rhythm. In the last case, the ID may be called defibrillator or cardioverter, instead. Alternative or additional functions of intracardiac devices may comprise providing other electrical or electromagnetic signals to the heart or its surrounding tissue, sensing electrical or electromagnetic signals or other physiological parameters of the heart and/or its surrounding tissue. In case, the ID is focused on sensing electrical or electromagnetic signals it may be alternatively called (bio)monitor. The ID may contain any combination of above functions. The implantation of the ID may comprise any fixation to the heart's tissue comprising the fixing within the atria and the ventricles of the heart or a fixing at the outer surface of the heart's tissue using small tines.

The inventive header assembly is suitable for an ID which usually comprises a cylindrical housing and the header assembly located at the distal end of the housing. Further, a pin-shaped electrode projects from the distal end of the housing, wherein the header assembly is arranged at and attached to the distal end of the housing of the ID such that the electrode projects through the header assembly, i.e. through a respective through-going or complete opening of the header assembly. The opening may be a central opening located at and along a longitudinal axis of the ID housing and the header assembly. The longitudinal axis forms the axial direction of the ID and the header assembly. The proximal cap, the base ring and the distal cap comprise the through-going opening, as well, wherein the size of the opening of the proximal cap may be such that an electrode feedthrough located at the proximal end of the electrode may be at least partly arranged within this opening. The cylindrical housing comprises the electronics module having a processor, an energy source (e.g. a battery or coil (for wireless charging)) and, if applicable, a communication component such as an antenna. The processor may be adapted to process signals/data determined from the patient's body or received from the surrounding environment and/or to produce signals for treatment of the patient's heart. Such signals may comprise electrical stimulation in the form of pulses in order to generate a physiologically appropriate heartrate, shocks for cardioversion or defibrillation in order to restore a more normal heart rhythm and/or other electrical or electromagnetic signals to the heart or its surrounding tissue. Such signals are transformed and transmitted by the electronic module and may be applied by the pin-shaped electrode to the heart or its surrounding tissue. The pin-shaped electrode is electrically connected to the electronics module and the energy source. The hermetically sealed housing may comprise electrically conducting material, e.g. titanium or stainless steel, and may function as another electrode. The header assembly comprises elements (the tines) for fixation of the ID to the selected tissue of the patient according to a treatment plan of a heath care provider (HCP), for example a ventricular wall of the patient's heart. Further, the header assembly provides electrical isolation of the pin-shaped electrode with regard to the tines and/or the ID housing. The cylindrical feedthrough provides a seat for the pin-shaped electrode and electrical isolation of it with regard to the housing.

The electrical isolation is particularly caused by the distal cap and the proximal cap, wherein the distal cap and the proximal cap comprise electrically isolating material, wherein the base ring is accommodated between the proximal cap and the distal cap in axial direction. The base ring carries at least two tines, for example two tines, four tines or six tines, protruding in distal direction from the base ring which provide fixation of the ID within the tissue of the patient at the desired treatment location after implantation. Therefore the tines are anchoring within the tissue.

For accommodation of the feedthrough and the electrode, the proximal cap, base ring and distal cap are all basically and/or essentially ring-shaped and accommodated in this consecutive order from proximal to distal direction along an axial direction, wherein the feedthrough and the pin-shaped electrode are located within the inner opening of the respective ring after completion of the manufacturing. Such uniaxial stackable assembly configuration from all rotational symmetrical components is advantageous because these components can be manufactured easily and at low cost. Further, they allow uniaxial assembly which is automated production friendly. The inventive header assembly construction as indicated above and below further avoids notches in the isolating components (distal cap and proximal cap) which reduces the complexity of the header assembly components as they are symmetrical, turned components having a longitudinal axis which also represents the axial direction.

The cylindrical feedthrough comprises an outer shell surface at least at its distal end. Further, the cylindrical feedthrough forms a distal end face. The distal end of the cylindrical feedthrough forming the outer shell surface is regarded a component of the header assembly.

After completion of the ID production and fixing of the header assembly to its end face the ring-shaped distal cap forms a permanent connection which counteracts a movement of the distal cap and the feedthrough apart from each other in distal direction. The connection is provided by a surface structure which is provided at the inner surface of a through hole of the distal cap and/or the outer shell surface of the feedthrough. According to the invention, the inner surface of the distal cap forms a locking connection with the outer shell surface of the feedthrough.

For example, according to an embodiment, the distal cap comprises a structure at the inner surface of its fixing portion which is configured for establishing a snap-fit locking connection with the outer shell surface of the feedthrough arrangement. In other words, the fixing portion of the distal cap and the feedthrough arrangement are adapted at their opposing surfaces such that, during assembling both components by pushing the distal cap axially onto the feedthrough arrangement, at least one of both components is temporarily deformed until reaching a position at which a snap-fit locking connection is established between both components. In such snap-fit locking connection, both components may engage at their opposing surfaces in a form-fit fashion. Therein, the opposing surfaces are in form-fit engagement without substantially permanently deforming one of the components, i.e. without substantial forces being exerted in a radial direction onto at least one of the fixing portion of the distal cap and the feedthrough arrangement.

More specifically, according to an embodiment, the outer shell surface of the feedthrough arrangement and the inner surface of the fixing portion of the distal cap have surface structures with protrusions and recesses being at least partially complementary to each other such as to establish a snap-fit locking connection between the outer shell surface and the inner surface. Thus, protrusions at the surface of one of the inner surface of the fixing portion of the distal cap and the outer shell surface of the feedthrough arrangement may engage into recesses at an opposing surface of the other component in a snap-fit manner.

According to an alternative exemplary embodiment, the fixing portion of the distal cap is configured for establishing a press-fit locking connection with the outer shell surface of the feedthrough arrangement. Similarly to the above described establishing of the snap-fit connection, the fixing portion of the distal cap and the feedthrough arrangement may be pushed together in an axial direction and may slide onto each other while being temporarily slightly radially deformed. Upon reaching a final position, the radial deformation may be partly released. However, a rest of such elastic deformation may remain and may result in a radial pressure being exerted between the inner surface of the fixing portion and the outer shell surface of the feedthrough arrangements. Due to such radial pressure, some permanent deformation may be induced in at least one of these surfaces. Accordingly, in such press-fit locking connection, substantive actual pressures may act between the connected components and, additionally, preconfigured surface structures and/or induced deformations at the opposed surfaces of the components may engage in a form-fit fashion.

More specifically, according to an embodiment, the outer shell surface of the feedthrough arrangement and the inner surface of the fixing portion of the distal cap have surface structures being at least partially non-complementary to each other such as to establish a press-fit locking connection between the outer shell surface and the inner surface. In other words, at least before being engaged, the inner surface of the fixing portion and the opposing outer shell surface of the feedthrough arrangement may both have protruding and/or recessed structures which, however, are not complementary to each other. Accordingly, upon being assembled, those opposing surfaces may not completely engage with each other without radial forces being locally induced at portions of the engaged surfaces which are non-complementary to each other. These radial forces and/or resulting permanent deformations at the engaged surfaces are typical for the press-fit locking connection.

Accordingly, the inner surface of the distal cap and the outer shell surface of the feedthrough may comprise a first surface structure adapted to provide a form locking connection with the respective other surface when the distal cap is attached to the feedthrough, wherein the form locking connection may comprise force locking, as well. The other surface is the inner surface of the distal cap or the other shell surface of the feedthrough. After assembly/fixing the feedthrough and the distal cap are permanently connected by a press-fit connection or snap-fit connection at their adjoining surfaces so that they cannot move relative to each other. The first surface structure of the outer shell surface of the feedthrough and/or of the inner surface of the distal cap or of both interact and engage and/or interlock with each other to form the press-fit or snap-fit connection. The first surface structure may comprise protrusions, for example extending in radial direction and forming an undercut, e.g. saw-tooth protrusions, a threaded structure, or may comprise a bayonet joint. A relative movement of the distal cap and the feedthrough is not possible in the fixed state (i.e. the fully assembled state), therefore also not a movement apart from each other in axial direction. Hence, gluing is avoided. Further, the production may use simple movement and forces directed in axial direction thereby avoiding more complicated rotational assembly movements.

The ring-shaped proximal cap is adapted to be fit into and along a respective circular recess of the distal end face of the ID housing in order to provide easy, exact and fast positioning during production. For that, the proximal cap may form a cylindrical protruding rim at its proximal surface.

Additionally, another form locking connection is provided for fixing the base ring between the distal cap and the proximal cap. This form locking connection is further described below.

The distal cap may comprise a stopper face at a distal section of its inner surface. The stopper face may be formed by a proximal surface of a protrusion projecting in radial direction from the inner surface of the distal cap, wherein the radial directions runs radially from the central longitudinal axis of the ID or its header assembly. The protrusion may be located at the most distal section of the inner surface of the distal cap. The stopper face interacts with the distal end face of the distal section of the feedthrough and forms a mechanical stop during assembling of the header assembly and the ID. The stopper face stops the press-fitting or snap-fitting movement of the distal cap or the ID housing at the correct position and thereby improves production quality. It further avoids mechanical damage of one of the press-fitted or snap-fitted components since it avoids mechanical overload by limiting the distance of movement of the components during press-fitting or snap-fitting.

An inner surface of the proximal cap formed by a through hole of the proximal cap may comprise a second surface structure and/or may form a form locking connection with the outer shell surface of the feedthrough when the proximal cap is attached to the feedthrough. In the same way as the distal cap the proximal cap may as well form a permanent press-fit or snap-fit, form locking connection with the outer shell surface of the feedthrough after completion of assembly. The forces acting in this essentially form locking connection may comprise force locking, too. The surface structure of the outer shell surface of the feedthrough or of the inner surface of the proximal cap or of both may interact and engage and/or interlock in the same manner as the distal cap and the feedthrough. Thereby gluing is avoided, as well, and production efficiency is enhanced.

The first surface structure and/or second surface structure may comprise at least two protrusions, wherein the at least two protrusions are accommodated in an axial direction one above the other and/or in circumferential direction next to each other, and/or a threaded profile. Preferably, the first surface structure and/or the second surface structure comprise a plurality of such protrusions accommodated one above the other or next to each other as described above. All protrusions project at least partially in radial direction from the surface forming the first or second surface structure, i.e. from the inner surface of the distal cap, from the inner surface of the proximal cap and/or from the outer shell surface of the feedthrough. Dimension of the protrusions in radial direction (perpendicular to the axial direction) may be less than 200 μm, preferably less than 150 μm (for example for a surface structure at the outer shell surface) in order to reliably fix the distal cap to the feedthrough. It may be greater than 50 μm. It was calculated by FEA that these dimensions of protrusions widen the distal cap diameter in such a way that the strain in the distal cap material (e.g. PEEK) reaches 50%-95% of its tensile strength (which is ca. 100 MPa at maximum). The inner surface of the distal cap and/or the inner surface of the proximal cap may comprise a threaded profile (female thread) and the outer shell surface may comprise a threaded profile (male thread) which is engaged in order to fix the distal cap and/or the proximal cap to the feedthrough. In one embodiment, the opposite threaded profiles form a self-locking thread.

The at least two protrusions may extend along at least part of the outer circumference of the outer shell surface of the feedthrough or extend along at least part of the inner circumference of the inner surface of the distal cap or of the inner surface of the proximal cap. This means that the at least two protrusions have a pre-defined length along the outer circumference or along the inner circumference. They may extend along ¼ of the respective circumference, along ½ of the respective circumference or along full circumference or even longer. The at least two protrusions may extend inclined with regard to the axial direction or perpendicular to this direction. The at least two protrusions may be distributed at the inner surface of the distal cap or the proximal cap or at the outer shell surface of the feedthrough or their length may be adapted such that the forces deriving from the press-fitting or snap-fitting of the feedthrough and the distal cap or the proximal cap, respectively, are well distributed across these surfaces in order to avoid stress peaks.

In one embodiment the respective other surface comprises at least one indentation for receiving the at least two protrusions when the distal cap or the proximal cap is attached to the feedthrough. For example, the outer surface of the feedthrough comprises at least two protrusions and the inner surface of the distal cap comprises at least one indentation which may mirror the at least two protrusions so that they perfectly interlock with each other after completion of assembling. The fixing may also be described as a snap-in step. For example, the inner surface of the distal cap may comprise a circular groove extending around the full circumference of the inner surface. In another embodiment the at least two pin-shaped protrusions extending from the outer shell surface of the feedthrough and L-shaped indentations of the inner surface of the distal cap form a bayonet connection. Having an indentation at the other surface reduces the strain in the distal cap or the proximal cap material, for example polymer material, thereby mitigating potential material breakages caused by high strain.

In one embodiment at least part of the at least two protrusions have a saw-tooth shape, for example, the at least two protrusions form at least two saw-tooth shaped circular rims accommodated one over the other in axial direction and extend along the full circumference or along part of the circumference, wherein the inclined surface of the saw-tooth shape has an angle, for example a small angle, e.g. with a value of more than or equal to 45°, preferably more than or equal to 60° but less than 90° with regard to the radial direction. With its angled shape the saw-tooth shaped protrusions ease the assembly by this slide-in chamfers. On the contrary the saw-tooth shaped rim with its second angle between 110° and 70°, preferably between 100° and 80°, with regard to the axial direction enclosed by the surfaces of the each protrusion which project from the outer shell surface of the feedthrough, “bites” into the distal cap inner surface and prevents the distal cap from becoming loose. A permanent fixation is thereby established. Alternatively or additionally, the at least two protrusions may form barbs at their furthest outwardly protruding end in order to further enhance their retention properties.

In one embodiment, the outer rim of the protrusions may have a circular cross section. In another embodiment the cross section of the outer rim of the protrusions may have a rounded polygonar form, e.g. a trilobular form. This gives the polymer distal cap space for inside deforming, reduces the stress to the distal cap and prevents it from breakage. Further, this solution is less prone to manufacturing tolerances because a wider range of cap inside diameters fits without breakage and/or may have self-locking behaviour.

In the following, some possible characteristics and advantages of the pressing arrangement of embodiments of the header assembly are described.

According to an embodiment, the pressing arrangement has a higher deformability in and against the radial direction than the fixing portion of the distal cap. Thus, upon radial forces being exerted between the base ring, on the one side, and the distal cap, on the other side, these forces also act onto the pressing arrangement. As the pressing arrangement has a higher deformability than the fixing portion of the distal cap, a smaller deformation is induced in the fixing portion as compared to the pressing arrangement as a result of such radial forces. Accordingly, any risk of environmental stress cracking is reduced in the fixing portion of the distal cap. As a result, a reliability of the distal cap's fixation to the feedthrough arrangement may be increased.

According to an embodiment, the pressing arrangement is an integrated portion of the distal cap. In other words, the pressing arrangement may not be provided as a separate component but may be an integrated part of the distal cap. Accordingly, no additional component has to be manufactured and/or handled during an assembling procedure.

For example, according to an embodiment, the distal cap comprises at least one lip portion protruding from the outer surface of the distal cap. The lip portion may be a protrusion which extends away from a main portion of the distal cap, such main portion including, inter alia, the fixing portion of the distal cap. The lip portion may be of a cantilever geometry. The lip portion may be ring-shaped. Particularly, the lip portion may extend coaxially or angled with regards to the main portion of the distal cap. The lip portion may have a substantially smaller thickness than the main portion of the distal cap. Due to its geometry and/or small thickness, the lip portion has a substantially larger deformability as compared to the main portion and particularly as compared to the fixing portion of the distal cap. Accordingly, the lip portion may be easily deflected or bent upon forces being applied onto the lip portion by the base portion.

According to a specific embodiment, the distal cap comprises an undercut recess extending adjacent to the outer surface in a direction parallel to an axial center axis of the distal cap, the undercut recess separating the lip portion from an inner portion of the distal cap. Such recess may separate an integral portion of the distal cap acting as a lip portion from a remaining portion of the distal cap including the fixing portion. A width of the recess may be broader than a thickness of the lip portion. Due to the recess extending in a direction parallel to the axial centre axis of the distal cap, the lip portion may then be easily deformed or deflected in a direction crossing the axial centre axis, i.e. in a direction towards the fixing portion of the distal cap. Upon being elastically deflected in such direction, the lip portion may apply a force onto the base ring, thereby pressing the base ring against the inner surface of the proximal cap. In this embodiment the main axis of the lip portion would be pointing in the proximal direction. Pointing in the proximal direction is to be understood as away from the radial direction towards the proximal end.

According to an alternative specific embodiment, the distal cap comprises an undercut recess extending adjacent to the outer surface in a direction crossing, or particularly being perpendicular to, an axial center axis of the distal cap, the undercut recess separating the lip portion from an upper portion of the distal cap. Again such recess may separate an integral portion of the distal cap acting as a lip portion from a remaining portion of the distal. A width of the recess may be broader than a thickness of the lip portion. Due to the recess extending in a direction crossing the axial centre axis of the distal cap, the lip portion may then be easily deformed or deflected in a direction parallel to the axial centre axis, i.e. for example in a direction away from the inner surface of the proximal cap. Upon being elastically deflected in such direction, the lip portion may apply a force onto the base ring, thereby pressing the base ring against the inner surface of the proximal cap.

According to another specific embodiment, the lip portion may be of a cantilever geometry pointing in the distal direction. Pointing in the distal direction is to be understood as away from the radial direction towards the distal end. The lip portion may be ring-shaped. Particularly, the lip portion may extend angled with regards to the main portion of the distal cap. The lip portion may have a substantially smaller thickness than the main portion of the distal cap. Due to its geometry and/or small thickness, the lip portion has a substantially larger deformability as compared to the main portion and particularly as compared to the fixing portion of the distal cap. Accordingly, the lip portion may be easily deflected or bent upon forces being applied onto the lip portion by the at least two tines. The lip portion may then be easily deformed or deflected in a direction crossing the axial centre axis, e.g. in a direction towards the axial centre axis. Upon being elastically deflected in such direction, the lip portion may apply a force onto the at least two tines and thereby to the base ring, thereby pressing the base ring against the inner surface of the proximal cap. This would be of particular advantage in a situation when the at least two tines are bend in the distal direction, e.g. when the implant is loaded into the implantation catheter.

The above disclosed embodiments describing a lip could be implemented alone or in combination with each other, in particular the distal cap may comprise a lip pointing in the proximal and/or a lip pointing in the distal direction.

According to a further alternative embodiment, the pressing arrangement comprises a pressing ring being interposed between the distal cap and the proximal cap such as to press onto the base ring. In such embodiment, the pressing arrangement is not implemented by an integral portion of the distal cap button by the separate pressing ring. Such pressing ring may be prepared and provided as a separate component and may be included into the header assembly in the assembling procedure. The pressing ring may be accommodated within the header assembly in an indent-feature such as a ring-like recess comprised for example in distal cap at or close to the inner surface. The pressing ring may consist of another material than the distal cap, particularly of a material having a higher elastic flexibility.

According to another embodiment, the base ring is configured such as to be non-circular in its non-deformed state. Such non-circular base ring may for example have an elliptic shape, a polygonal shape, etc. Particularly, such base ring, in its undeformed state, may have portions having a smaller diameter than the outer surface of the distal cap and/or portions having a larger diameter than the inner surface of the proximal cap. Accordingly, upon being interposed between the distal cap and the proximal cap, such non-circular base ring may be elastically deformed such as to conform with the circular outer surface of the distal cap and/or the circular inner surface of the proximal cap. Due to such deformation, the base ring will exert forces onto at least one of the distal and proximal caps, these forces resulting in an intended friction force between the base ring and the respective cap.

In the following, some further possible characteristics and advantages of the header assembly are described.

In one embodiment the inner surface of the distal cap is inclined or tapered, wherein an inner diameter of a most proximal section is greater than an inner diameter of a section distally from the most proximal section. Alternatively, an inner diameter of a most proximal section is smaller than an inner diameter of a section distally from the most proximal section. If there are protrusions at the inner surface of the distal cap or at the outer shell surface of the feedthrough, their inner or outer diameter may increase or decrease along axial direction, accordingly. If the inner diameter of the surface or the protrusions increases in proximal direction along axial direction the retention force of the connection of the distal cap and the feedthrough increases. However, the stress to the material of the distal cap increases, too.

In one embodiment the distal cap and the proximal cap comprise electrically isolating material and the distal cap and/or the proximal cap additionally may comprise elastic material. The distal cap and/or the proximal cap may comprise or may fully be composed of polyether ether ketone (PEEK), liquid crystal polymer (LCP), polysulfone (PSU) or other polymer material with similar properties. The elasticity of the above materials is advantageous for the manufacturing process as it helps to establish the press-fitting connection.

In one embodiment the header assembly may comprise a ring shaped steroid depot which is accommodated in axial direction between the distal cap and the distal end face of the feedthrough. The steroid depot contains at least one medical substance, for example an anticoagulant and/or an antibacterial substance. The medical substance may be released gradually into the blood close to the fixation location of the ID within the patient's tissue in order to heal the damaged tissue close to the fixation location. The steroid depot may be clamped between the stopper face of the distal cap and the distal end face of the feedthrough so that it is permanently fixed at the header assembly and the ID. Further, an inner rim protruding in distal direction may be located adjacent to a respective stopping face of the pin-shaped electrode located at the proximal end of the pin head. Thereby, the electrode keeps the steroid depot in place.

In one embodiment, the form locking fixing of the base ring with the tines between the proximal and distal caps is provided by conically formed surfaces at the proximal and distal cap and a conical form of the base ring. The conical form of the base ring means that the inner and the outer surface of the ring have a conical, inclined form, wherein both surfaces run essentially parallel. In particular, the conical form of the base ring means that the inner diameter and the outer diameter of the base ring is greater at its distal end than the respective diameter at its proximal end. If both sides of the base ring run parallel, the wall thickness of the base ring is constant along its entire axial length. In another embodiment, its wall thickness may change along its length (i.e. become thinner or thicker along the axial direction and into distal direction). The base ring with the at least one tine is clamped and fixed between the proximal cap (on its proximal side) and the distal cap (on its distal side). For that a side face (distal face) of the proximal cap adjacent the base ring and a side face (proximal face) of the distal cap adjacent the base ring have the same inclination or slope as the respective lateral surface of the base ring. This optimizes space and results in fewer header components causing less processing and assembly steps with less costs during manufacturing of the ID. The axial length and volume of the header is minimized. This improvement allows more space for other more critical features of the device, such as the battery, which would increase device longevity. The base ring is conically shaped to allow for axial height reduction while maintaining band height. In other words, the space could be allotted to the electronics module, to incorporate more therapeutic features. Conversely, for the same battery and electronics module size, a reduction in header length would allow for a reduction in overall device length. This enables application for smaller patients, or alternate placement within the heart, such as the right atrium.

In one embodiment, each of the at least two tines comprises an abutting section directly extending from the base ring and forming a connection with the base ring and a flex zone, wherein the abutting section of the respective tine continues the conical form of the base ring. Each of the plurality of tines terminates into the base ring tangent to the arc of the tines just below the surface of the distal cap and the base ring is contained fully by the distal cap at its distal side and by the proximal cap at its proximal side. The middle section of each tine of the plurality of tines has a curved form (e.g. circular curved) and the end section furthest from the base ring comprises a straight section. Other forms of each tine are possible, as well. In one embodiment the base ring and the at least one tine are integrally formed. The base ring and/or the at least two tines may partially or fully consist of biocompatible material, e.g. shape memory material, for example Nitinol.

According to a second aspect of the present invention, an implantable intracardiac device is described, the device having a cylindrical housing and a header assembly realized as described above, wherein the feedthrough is accommodated at the distal end of the housing, wherein the feedthrough is integrally formed with the housing or is formed by a separate element which is fixed and hermetically sealed at the distal end face of the housing, for example by welding.

According to a third aspect of the present invention, a manufacturing method for a header assembly as described above is described, wherein the manufacturing method comprises the following steps:
Providing the feedthrough, the proximal cap, the distal cap and the base ring with the at least two tines,
Arranging the proximal cap, the base ring and the distal cap one above the other (i.e. in this consecutive order) in axial direction such that the base ring is arranged between the proximal cap and the distal cap,
Fixing the proximal cap, the base ring and the distal cap to the feedthrough by application of an axial force onto a distal face of the distal cap and/or onto a proximal section of the feedthrough such that the base ring is fixed in an axial direction between the distal cap and the proximal cap and is rotatable around the axial direction relative to the distal cap, that the feedthrough is accommodated within a through-hole of the distal cap and that a surface structure of that at least one of the inner surface of the distal cap and the outer shell surface of the feedthrough provides one of a snap-fit and a press-fit locking connection with the respective other surface in the fixed state, wherein in the fixed state the surface structure forming the locking connection counteracts a movement of the distal cap and the feedthrough apart from each other in axial direction.

The axial force may be provided by a pressing tool applying an axial force in distal direction to the distal end face of the distal cap and/or in proximal direction to the proximal section of the feedthrough. By the applied axial force the friction forces between the distal cap inner surface and the feedthrough outer shell surface are overcome so that the above described snap-fit or press-fit connection of the distal cap and the feedthrough is established thereby counteracting a movement of the distal cap and the feedthrough apart from each other in axial direction. The housing/feedthrough is supported/fixed during this snap-fit or press-fit-process. The same applies to the connection of the proximal cap and the feedthrough, if a snap-fit or press-fit connection is also established between these components.

According to a fourth aspect of the present invention, a manufacturing method for an implantable intracardiac device as described above is described, wherein the manufacturing method comprises the following steps:
Providing the cylindrical housing with electric or electromagnetic components within the housing, the cylindrical feedthrough at the distal end of the housing, integrally formed with the housing or as separate element fixed at and hermetically sealed at the distal end face of the housing, and a pin-shaped electrode protruding from the distal end of the feedthrough and fixed within a recess of the feedthrough,
Providing the proximal cap, the distal cap and the base ring with the at least two tines,
Arranging the proximal cap, the base ring and the distal cap one above the other in axial direction such that the base ring is arranged between the proximal cap and the distal cap,
Fixing the proximal cap, the base ring and the distal cap to the feedthrough by application of an axial force onto a distal face of the distal cap and/or onto a proximal section of the feedthrough such that the base ring is fixed in an axial direction between the distal cap and the proximal cap and is rotatable around the axial direction relative to the distal cap, that the feedthrough is accommodated within a through-hole of the distal cap and that a surface structure of that at least one of the inner surface of the distal cap and the outer shell surface of the feedthrough provides one of a snap-fit and a press-fit locking connection with the respective other surface in the fixed state, wherein in the fixed state the surface structure forming the locking connection counteracts a movement of the distal cap and the feedthrough apart from each other in axial direction.

In one embodiment the ring-shaped steroid depot is arranged in axial direction proximally from the distal cap prior fixing, wherein the ring-shaped steroid depot is fixed between the distal cap and a distal end face of the feedthrough in the fixed/assembled state.

In one embodiment the proximal cap is fixed within a recess of the distal surface of the housing, wherein the recess is circumferentially surrounded by an outer rim extending from the distal end face of the housing. The distal cap is the element which is arranged at the distal end of the header assembly but it may extend through the through-hole of the proximal cap and may thereby form a section of the proximal end face of the header assembly as well. In this case, the proximal cap and the distal cap form an end face of the header assembly accommodated adjacent the distal end face of the housing. Alternatively, only the proximal cap forms the proximal end face of the header assembly. In this other case, only the proximal cap is accommodated adjacent the distal end face of the housing.

In another step, the pin-shaped electrode may be fixed within a through hole of the feedthrough, wherein the proximal end face of the electrode head abuts to the steroid depot in the fixed state. The electrode is brazed into the ceramic of the feedthrough by a gold braze, that holds and hermetically seals the pin in the feedthrough ceramic. As an alternative the electrode is fixed inside the feedthrough by a glass to metal connection. The electrode can also be a two piece part consisting of a pin and an electrode tip. The two parts are for example welded together by means of laser welding

As described herein, the fixing of the essential header components is provided by a method not using adhesive bonding forces but using the elastic and plastic material properties of the polymer (e.g. thermoplastic) distal cap to achieve a reliable long term stable connection to the ID housing. Stretching the diameter of the distal cap to a certain degree that it does not break in combination with the surface structure (e.g. at the feedthrough outer shell surface) proves this snap-fit or press-fit connection as permanent attachment.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein

FIG. 1 shows a first embodiment of an inventive implantable ID with an inventive header assembly in a longitudinal-sectional, exploded and perspective view,

FIG. 2 depicts the embodiment of FIG. 1 in a longitudinal-sectional and perspective view,

FIG. 3 shows the embodiment of FIG. 1 in a longitudinal-sectional view,

FIG. 4 depicts an enlarged section of the housing with the feedthrough and the electrode of the embodiment of FIG. 1,

FIG. 5 outlines a manufacturing step of the embodiment of FIG. 1 in a longitudinal-sectional view,

FIG. 6 shows a second embodiment of an inventive intracardiac device with an inventive header assembly in a longitudinal-sectional view during manufacturing,

FIG. 7 shows the embodiment of FIG. 6 in a longitudinal-sectional view after finishing the manufacturing steps, and

FIG. 8 shows a third embodiment of an inventive implantable ID with an inventive header assembly in a longitudinal-sectional view.

FIG. 9 shows a fourth embodiment of an inventive implantable ID with an inventive header assembly in a longitudinal-sectional view.

FIG. 10 depicts a cross sectional view of a feedthrough and its inner components of a fifth embodiment of an inventive header assembly or intracardiac device, respectively.

FIG. 11 shows a sixth embodiment of an inventive implantable ID with an inventive header assembly in a cross-sectional view.

FIGS. 12A-B shows a seventh embodiment of an inventive implantable ID with an inventive header assembly in a cross-sectional view, whereby the tines are shown in an unrestricted configuration (FIG. 12A) and in a configuration as upon loaded into a implantation catheter (FIG. 12B)

### DETAILED DESCRIPTION

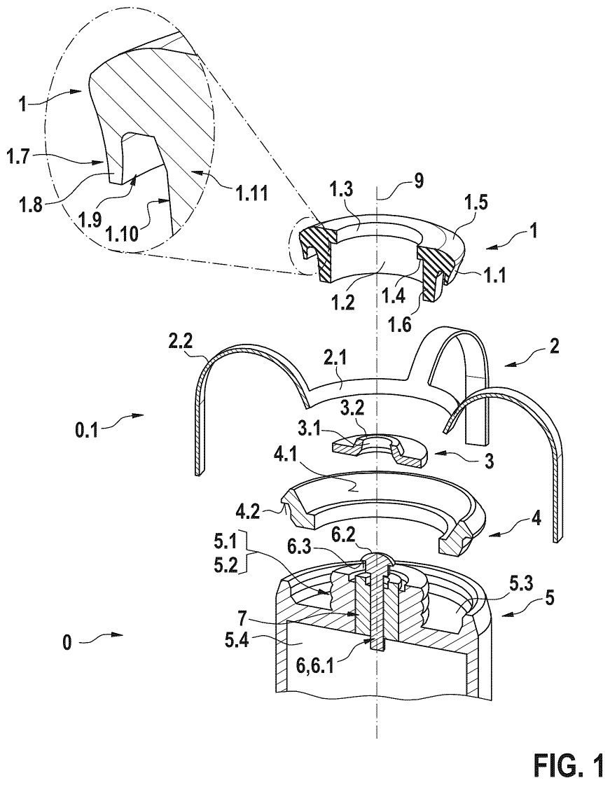

FIGS. 1 to 5 illustrates an exploded view of the components of the first embodiment of an implantable ID (0), e.g. a leadless pacemaker, with the header assembly (0.1). The components are a ring-shaped distal cap 1, a base ring assembly 2 comprising the base ring 2.1 and the four tines 2.2, a washer-like steroid depot 3, a ring-shaped proximal cap 4 and an ID housing 5 comprising a cylindrical distal section forming a feedthrough 5.1. Further, a pin-shaped electrode 6 extending therefrom in distal direction. The base ring 2.1 is conically formed in such way that a distal end of the base ring 2.1 has a greater inner and outer diameter compared with these diameters at its proximal end.

The distal cap 1, the base ring 2.1, the steroid depot 3 and the proximal cap 4—each of these components comprises a central through-going opening for accommodation of the electrode 6. The components referred to in the previous sentence are axially symmetrical with regard to the longitudinal axial centre axis 9 defining the axial direction. The diameter of the central opening of the distal cap 1, the base ring 2.1 and the proximal cap 4 is such that the electrode feedthrough 5.1 is located within this opening in the fixed/assembled state. The diameter of the electrode feedthrough 5.1 is greater than the diameter of the electrode 6.

The ring-shaped distal cap 1 comprises the through-going opening forming an inner surface 1.2 at a fixing portion 1.6. At the distal end of this opening a rim-shaped protrusion 1.3 is provided extending in radial direction from the inner surface 1.2 and forming a circular stopper face 1.4. Further, the distal cap 1 comprises an outer (proximal) conical, inclined surface 1.1 to which the inclined base ring 2.1 abuts in the assembled state. The distal cap 1 consists of electrically isolating and elastic material, for example PEEK.

The four tines 2.2 extend from the conical shaped base ring 2.1, wherein each tine 2.2 has an abutting section (flex zone) which transitions to the base ring 2.1, a curved middle section and a straight end section (furthest from the base ring 2.1). The tines 2.2 provide the mechanical fixation of the ID within the patient's heart after deployment and penetration of the heart's tissue such that the central electrode 6 is in mechanical and electrical contact with the inner tissue of the patient's heart within one ventricle or atrium. The proximal cap 4 ensures electric isolation of the tines 2.2 from the housing 5. The base ring assembly 2 consists of Nitinol, for example.

The header assembly 0.1 comprises a pressing arrangement 1.7. In the first and second embodiments shown in FIGS. 1-7, the pressing arrangement 1.7 is provided by a lip portion 1.8 protruding at an outer surface 1.10 of the distal cap 1. The lip portion 1.8 is an integral portion of the distal cap 1. The pressing arrangement 1.8 has a higher deformability in and against a radial direction perpendicular to the axial centre axis 9 than the fixing portion 1.6 of the distal cap 1. Specifically, the distal cap 1 comprises an undercut recess 1.9 extending adjacent to the outer surface 1.10 in a direction parallel to the axial centre axis 9 of the distal cap 1 (i.e. vertically). The undercut recess 1.9 separates the lip portion 1.8 from an inner portion 1.11 of the distal cap 1, the inner portion 1.11 including the fixing portion 1.6.

Having such specific configuration, the pressing arrangement 1.7 is configured to exert an elastic force in a radial direction, i.e. orthogonal to the axial centre axis 9, such as to press the base ring 2.1 against the inner surface 4.1 of the proximal cap 4, upon the base ring 2.1 being interposed between the proximal cap 4 and the distal caps 1. Accordingly, in such assembled configuration, the pressing arrangement 1.7 induces friction forces acting onto the base ring 2.1 upon the base ring 2.1 being rotated around the axial direction 9 relative to the caps 4, 1. Due to its high local deformability, the pressing arrangement 1.7 may be deflected upon assembling the header assembly and, as a result of such elastic deflection, the pressing arrangement 1.7 may then reliably press the base ring 2.1 against the inner surface 4.1 of the proximal cap 4 while the inner portion 1.11 of the distal cap 1 is not significantly deformed.

Accordingly, even in an assembled state with the base ring 2.1 being compressed between the distal cap 1 and the proximal cap 4, no significant permanent mechanical stresses are exerted onto the inner portion 1.11 and particularly the fixing portion 1.6 of the distal cap 1. Accordingly, a risk of any environmental stress cracking (ESC) occurring at the inner portion 1.11 of the distal cap 1 may be minimized. Thus, a locking connection formed between the outer shell surface 5.2 of the feedthrough arrangement 5.1 and the inner surface 1.2 of the distal cap 1 as described further below is not compromised due to ESC. Instead, permanent mechanical stress is only applied at the pressing arrangement 1.7. However, even in cases where such stress results in ESC at the pressing arrangement 1.7, the mechanical connection between the header assembly 0.1 and the housing 5 of the intracardiac device 0 is still reliably maintained.

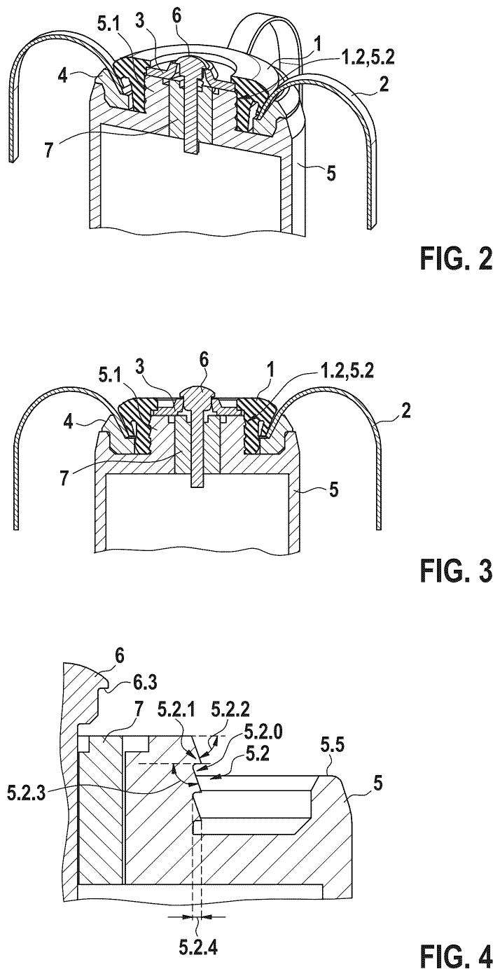

There is the washer-like steroid depot 3 comprising a through hole 3.1. The steroid depot 3 is made of a mixture of silicone and dexamethasone acetate. The inner section of the steroid depot 3 is slightly arched upwardly into distal direction forming a distally projecting rim 3.2 to which a stopper face 6.3 of the electrode head 6.2 abuts (see FIGS. 2 and 3).

The header assembly and the ID further comprise the proximal cap 4 forming an inclined surface 4.1 at its distal side. If one views the proximal cap 4 from the proximal direction the proximal cap comprises a circular stop surface 4.2 for abutting a rim 5.5 (see FIG. 4) at the distal end face of the housing 5. The circular rim 5.5 together with a circular recess 5.3 surrounding the feedthrough 5.1 cause centering of the proximal cap 4 and the distal cap 5. The proximal cap consists of electrically isolating and elastic material, for example PEEK.

As indicated above, the ID housing 5 forms the feedthrough 5.1 at its distal end. In the depicted embodiment the feedthrough 5.1 is integrally formed with the housing 5 but may alternatively be formed as a separate element which is hermetically sealed attached to the housing 5. The feedthrough 5.1 forms an outer shell surface 5.2 with surface structures 5.2.0 having a plurality of saw-tooth protrusions 5.2.1 which are shown in more detail in FIG. 4. Each protrusion forms a circular rim extending fully around the outer shell surface 5.2 and forming a first inclined surface and a second inclined surface. The first angle 5.2.2 is confined by the first inclined surface and the radial direction (the radial direction runs perpendicular to the longitudinal axis 9) and a second angle 5.2.3 is confined by the second inclined surface and the axial direction. Further, the height of the protrusion in radial direction is depicted with reference number 5.2.4. The first angle 5.2.2 may be greater than or equal to 45°, for example 70°. The second angle 5.2.3 may be equal to 110° or less, preferably greater than or equal to 70°, for example 90°. The height may be chosen to be less than 200 μm, preferably between 50 μm and 150 μm. The protrusions 5.2.1 are formed such that their outer diameter is less at its distal end and greater at its proximal end. Thereby the distal cap 1 which inner surface 1.2 interacts with the protrusions 5.2.1 during press fitting movement during manufacturing can easily be slid along the outer shell surface 5.2 in proximal direction but cannot be removed in the opposite, distal direction because the protrusions 5.2.1 “bite” into the inner surface 1.2 of the distal cap 1.

The housing 5 of the intracardiac device contains in its inner volume 5.4 a battery and an electronic module comprising a processor and ensures hermetically sealing of these components. These components are electrically connected to the electrode 6 and provide the electrical stimulation of the heart or processing of electrical signals determined from the heard. Further, the housing may contain components for communication such as an antenna. The housing may consist of a titanium alloy or stainless steel.

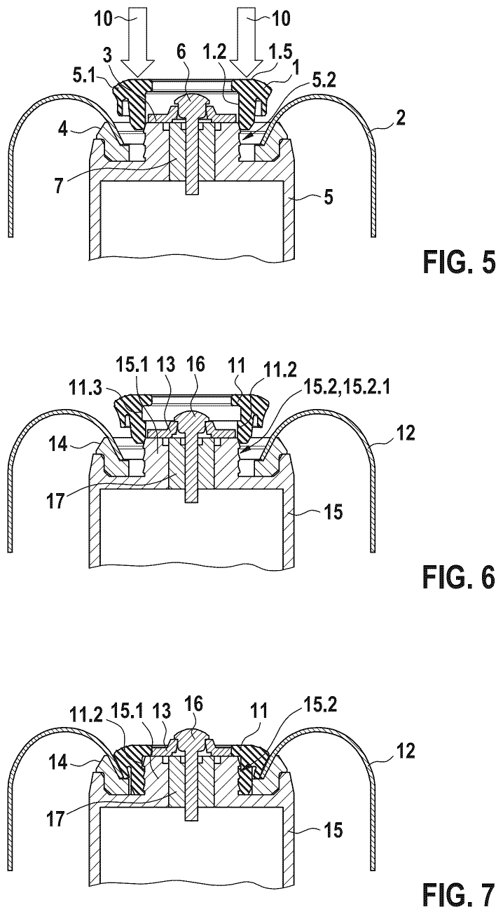

As shown in FIG. 5, during manufacturing first the proximal cap 4 is accommodated at the distal end of the housing 5 so that its stopper face 4.2 is adjacent the distal rim 5.5. Further, the base ring 2.1 is provided such that its outer conical surface abuts the inclined distal surface 4.1 of the proximal cap 4. The base ring 2.1 is accommodated distally from the proximal cap 4. Additionally, the electrode 6 is provided with its proximal shaft 6.1 within a through hole of the feedthrough where it is electrically isolated from the housing 5 by a hollow cylindrical isolator 7 but electrically connected with the electrical components located within the housing 5. The steroid depot 3 is clamped between the distal end face of the feedthrough 5.1 and the proximal stop face 6.3 at the head 6.2 of electrode 6, wherein the distal rim 3.2 of the steroid depot 3 abuts the stop face 6.3.

As shown in FIG. 5, the last manufacturing step is the press fitting step provided by distal movement of the distal cap 1 and applying an axial force at the distal surface 1.5 of the distal cap, for example by using a stamp (depicted by arrows 10), thereby sliding the inner surface 1.2 of the distal cap 1 along the outer shell surface 5.2 of the feedthrough 5.1 until the stopper face 1.4 abuts the distal surface of the steroid depot 3. The interaction method is a press-fit connection using the elastic and plastic material behavior of the PEEK material of the distal cap 1 to create a permanent fixation between the feedthrough flange (feedthrough outer shell surface 5.2) and the distal cap 1. The press-fit connection is achieved by overcoming friction forces between the distal cap 1 inner surface 1.2 and the saw-tooth protrusions 5.2.1. The housing 5/feedthrough outer shell surface 5.2 is supported/fixed during this press-fitting process. As indicated above the protrusions 5.2.1 “bite” into the inner surface 1.2 of the distal cap 1 thereby forming a permanent mechanical connection between the distal cap 1 and the feedthrough 5.1 counteracting a movement of the distal cap 1 and the feedthrough 5.1 apart from each other in axial direction. Thereby the base ring assembly 2 and the proximal cap 4 as well as the steroid depot 3 are permanently fixed, as well.

In this embodiment the proximal cap 4 does not interact with the outer shell surface 5.2 of the feedthrough 5.1. In an alternative embodiment, the inner surface of the distal cap 1 may be shorter and the inner surface of the proximal cap 4 interacts with the outer shell surface 5.2 of the feedthrough 5.1 in the same manner as the distal cap 4. For that, the inner diameter of the proximal cap 4 is less than for the embodiment depicted in FIG. 1 to 5.

The first embodiment of FIG. 1 to 5 comprises a distal cap 1 which does not have any indentation at its inner surface 1.2. The second embodiment shown in FIGS. 6 and 7 differs from the first embodiment in this regard. The reference numbers of the elements of the second embodiment correspond to the reference numbers of the respective elements of the first embodiment with the number ten added.

The inner surface 11.2 of the distal cap 11 comprises a circular groove 11.3 forming an indentation which interlocks with the distal saw-tooth protrusions 15.2.1 at the outer shell surface 15.2 of the feedthrough 15.1 during and after assembly. This may reduce the strain in the distal cap 11 polymer material mitigating potential material breakages caused by high strain.

In the first and second embodiments, the inner surface 1.2 at the fixing portion 1.6 of the distal cap 1 is configured such as to form a locking connection with the outer shell surface 5.2 of the feedthrough arrangement 5.1 for counteracting a movement of the distal cap 1 and the feedthrough arrangement 5.1 apart from each other in an axial direction. In this embodiment, the inner surface 1.2 and the opposing outer shell surface 5.2 with the surface structures 5.2.0 are configured such as to form the locking connection as a press-fit connection. Therein, the surface structures 5.2.0 at the shell surface 5.2 of the feedthrough arrangement 5.1 and the inner surface 1.2 of the fixing portion 1.6 are at least partially non-complementary to each other such that, upon pushing the distal 1 onto the feedthrough arrangement 5.1, the press-fit locking connection is established between both opposing surfaces.

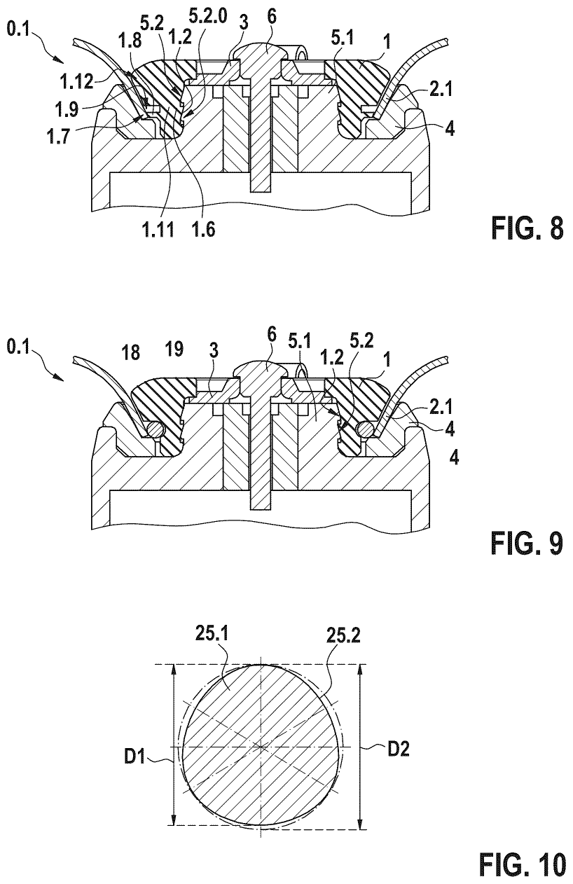

However, in alternative embodiments as depicted in FIGS. 8 and 9, the inner surface 1.2 and the outer shell surface 5.2 may also comprise surface structures 5.2.0 with protrusions and recesses being at least partially or preferably fully complementary to each other such as to establish a snap-fit locking connection between the outer shell surface 5.2 and the inner surface 1.2. Therein, during an assembly procedure, the distal cap 1 is pushed onto the feedthrough arrangement 5.1 but only temporarily deforms (i.e. widens) in a radial direction upon the protrusions and recesses sliding along each other. However, upon reaching a final configuration, the protrusion engage with the recesses in a complementary manner such that no substantial permanent stress or deformations are induced between the distal cap 1 and the feedthrough arrangement 5.1.

In the third embodiment shown in FIG. 8, the pressing arrangement 1.7 comprises a lip portion 1.8 extending in the radial direction (i.e. horizontally). The lip portion 1.8 is formed by an undercut recess 1.9 extending adjacent to the outer surface 1.10 in a direction crossing the axial centre axis 9 of the distal cap 1 (i.e. horizontally or diagonally). Accordingly, the undercut recess 1.9 separates the lip portion 1 8 from an upper portion 1.12 of the distal cap 1.

Upon the header assembly being assembled with the base ring 2.1 being interposed between the proximal cap 4 and the distal cap 1, the lip portion 1.8 is slightly elastically deflected and thereby presses onto the base ring 2.1 and induces a friction upon the base ring 2.1 being rotated around the axial direction 9. However, no substantial permanent mechanical stress is applied to the inner portion 1.11 and the fixing portion 1.6 of the distal cap 1, thereby preventing any local environmental stress cracking and therefore preventing compromising a reliable connection of the header assembly to the rest of the ID.

In the fourth embodiment shown in FIG. 9, the pressing arrangement 1.7 comprises a separate pressing ring 18 such as a silicon O-ring. The pressing ring 18 is a separate component and is interposed between the distal cap 1 and the proximal cap 4. Particularly, the pressing ring 18 may be accommodated and held in a ring-like indent 19 comprised at the outer surface of the distal cap 1. The pressing ring 18 is made from a material having a higher deformability than the material of the distal cap 1. Accordingly, upon the header assembly being assembled with the base ring 2.1 and the pressing ring 18 being interposed between the proximal cap 4 and the distal cap 1, the pressing ring 18 is elastically compressed and thereby presses onto the base ring 2.1. Thus, no substantial permanent mechanical stress locally occurs in the distal cap 1 due to any substantial deformations, thereby preventing ESC and finally preventing compromising a reliable connection of the header assembly to the rest of the ID.

FIG. 10 refers to a third embodiment of the header assembly/ID. The embodiment of FIG. 8 differs from the first embodiment in the cross section of the saw-tooth protrusions 5.2. The first embodiment of an inventive header assembly and ID comprises a circular cross section of the protrusions 25.2. The embodiment shown in FIG. 8 has protrusions which do not have a circular cross section but a rounded polygon for, e.g. a trilobular form. Such form is shown in FIG. 10.

It is noted that the sketch of FIG. 10 does not show the components of the header assembly within the feedthrough 25.1 in detail.

In another embodiment (not shown) the diameters of the protrusions at the outer shell surface of the feedthrough may differ from one to another. They may have all the same protrusion type but realize an overall angled shape comprising an increasing or decreasing outer diameter from one to the next protrusion (e.g. saw-tooth protrusion). Increasing diameters starting from the distal end increasing to the proximal end will increase the retention force but also increase the stress to the polymer material of the distal cap.

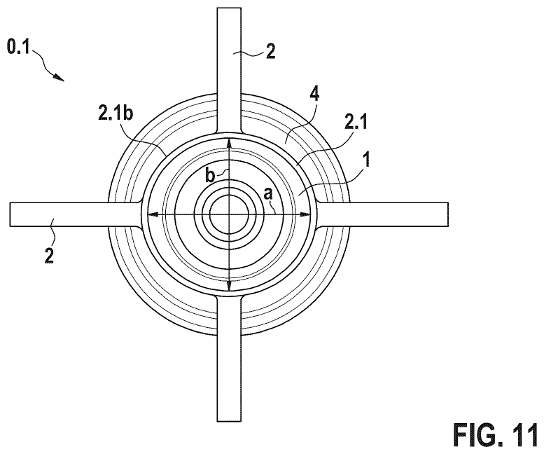

Another embodiment of the header assembly 0.1 is shown in FIG. 11. Therein, the pressing arrangement 1.7 is realised by using a base ring 2.1 which is non-circular in its non-deformed state. In other words, in its non-deformed state, the base ring 2.1 has a first diameter “a” in a first direction and a second diameter “b” in a second direction perpendicular to the first direction. The first and second diameters are different from each other, e.g. b<a. Accordingly, when such non-circular base ring 2.1 is interposed between the circular conical outer surface 1.1 of the distal cap 1 and the opposing circular conical inner surface 4.1 of the proximal cap 4, its non-circular geometry is slightly elastically deformed such as to become approximately circular. Thereby, elastic pressing forces are applied between the base ring 2.1 and the opposing caps 1, 4. These pressing forces result in friction forces upon the base ring 2.1 being rotated relative to the caps 1, 4. However, no substantial deformations are induced to the distal cap 1 such that its locking connection to the feedthrough arrangement is not compromised by any occurrence of stress induced cracking.

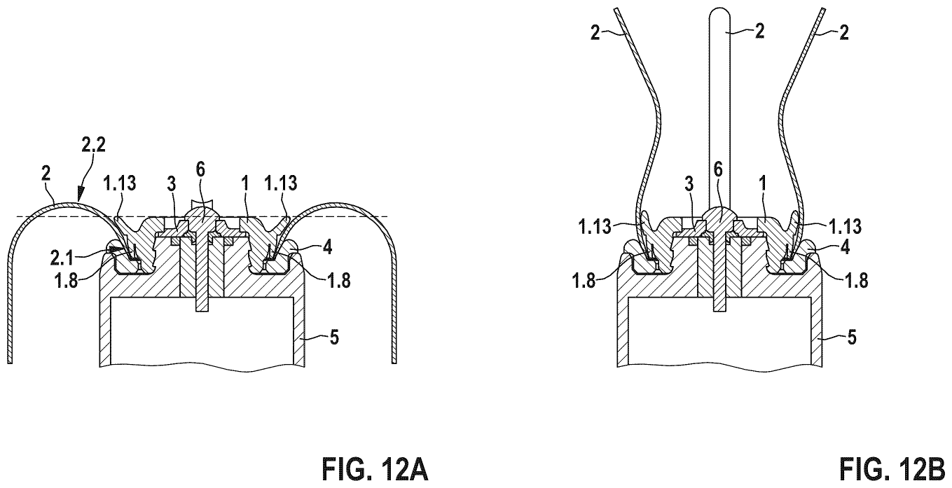

FIGS. 12A-B refers to a seventh embodiment of the header assembly/ID, whereby the tines are shown in an unrestricted configuration (FIG. 12A) and in a configuration as upon loaded into an implantation catheter (FIG. 12B). The header assembly according to this embodiment comprises a second lip portion 1.13 in addition to the lip portion 1.8 of the first and second embodiment shown in FIGS. 1-7. The second lip portion 1.13 has a cantilever geometry and points in the distal direction. If the tines 2 bends distally (FIG. 12B) due to loading of the implant into the implantation catheter (not shown), the tine array is kept attached to the device by the second lip at the distal cap 1 as well, creating an overhang surface such that the tine ring cannot escape the header assembly. Furthermore, the tines 2 have contact to the second lip 1.13 supporting the tine elastically beyond the original height of the header and thereby providing a gradual transition of the stiffness of the catheter.

The above embodiments have the following advantages:
No gluing is necessary during manufacturing because it realizes a press-fit permanent connection instead.
The manufacturing process is automated assembly friendly (uniaxial assembly).
The construction of the inventive header assembly reduces the header height.
No notches are necessary with regard to the isolating parts, namely the distal cap and the proximal cap thereby reducing the complexity of header components (all symmetrical, turnable components).
Low permanent stress fixating upper, distal cap, therefore mitigating environmental stress cracking in the polymer.
Preventing tine rotation by friction, in the same instance having a safety clutch mechanism when excessive strain should be applied to the tines.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A header assembly for an implantable intracardiac device, wherein the header assembly comprises:
a cylindrical feedthrough arrangement,
a ring-shaped proximal cap,
a ring-shaped distal cap,
a base ring with at least two tines protruding distally from the base ring, and
a pressing arrangement,
wherein the feedthrough arrangement has an outer shell surface,
wherein the proximal cap comprises an inner surface,
wherein the distal cap comprises an outer surface,
wherein the distal cap comprises a fixing portion with an inner surface,
the inner surface forming a locking connection with the outer shell surface of the feedthrough arrangement for counteracting a movement of the distal cap and the feedthrough arrangement apart from each other in an axial direction,
wherein the proximal cap, the distal cap and the base ring are configured such that the inner surface of the proximal cap and the outer surface of the distal cap are arranged coaxially and directed towards each other, and the base ring is interposed between the inner surface of the proximal cap and the outer surface of the distal cap such as to be coaxially rotatable relative to the distal cap,
wherein the pressing arrangement is configured such as to exert an elastic force in a radial direction such as to press the base ring against one of the inner surface of the proximal cap and the outer surface of the distal cap.

2. The header assembly according to claim 1,
wherein the distal cap comprises a structure at the inner surface of its fixing portion which is configured for establishing a snap-fit locking connection with the outer shell surface of the feedthrough arrangement.

3. The header assembly according to claim 1,
wherein the outer shell surface of the feedthrough arrangement and the inner surface of the fixing portion of the distal cap have surface structures with protrusions and recesses being at least partially complementary to each other such as to establish a snap-fit locking connection between the outer shell surface and the inner surface.

4. The header assembly according to claim 1,
wherein the fixing portion of the distal cap is configured for establishing a press-fit locking connection with the outer shell surface of the feedthrough arrangement.

5. The header assembly according to claim 1, wherein the outer shell surface of the feedthrough arrangement and the inner surface of the fixing portion of the distal cap have surface structures being at least partially non-complementary to each other such as to establish a press-fit locking connection between the outer shell surface and the inner surface.

6. The header assembly according to claim 1,
wherein the pressing arrangement has a higher deformability in and against the radial direction than the fixing portion of the distal cap.

7. The header assembly according to claim 1,
wherein the pressing arrangement is an integrated portion of the distal cap.

8. The header assembly according to claim 7,
wherein the distal cap comprises at least one lip portion protruding from the outer surface of the distal cap.

9. The header assembly according to claim 8,
wherein the distal cap comprises an undercut recess extending adjacent to the outer surface in a direction parallel to an axial center axis of the distal cap, the undercut recess separating the lip portion from an inner portion of the distal cap.

10. The header assembly according to claim 8,
wherein the distal cap comprises an undercut recess extending adjacent to the outer surface in a direction crossing an axial center axis of the distal cap, the undercut recess separating the lip portion from an upper portion of the distal cap.

11. The header assembly according to claim 8, wherein the distal cap comprising a lip portion having a cantilever geometry pointing in the distal direction.

12. The header assembly according to claim 1, wherein the pressing arrangement comprises a pressing ring being interposed between the distal cap and the proximal cap such as to press onto the base ring.

13. The header assembly according to claim 1, wherein the base ring is configured such as to be non-circular in its non-deformed state.

14. An implantable intracardiac device with a cylindrical housing and a header assembly according to claim 1, wherein the feedthrough arrangement is arranged at a distal end of the housing, wherein the feedthrough arrangement is integrally formed with the housing or is formed by a separate element which is fixed and hermetically sealed at a distal end face of the housing.

15. A manufacturing method for a header assembly according to claim 1, comprising the following steps:
Providing the feedthrough arrangement the proximal cap, the distal cap and the base ring with the at least two tines,
Arranging the proximal cap, the base ring and the distal cap one above the other in axial direction such that the base ring is arranged between the proximal cap and the distal cap,
Fixing the proximal cap, the base ring and the distal cap to the feedthrough arrangement by application of an axial force onto a distal face of the distal cap and/or onto a proximal section of the feedthrough such that the base ring is fixed in an axial direction between the distal cap and the proximal cap and is rotatable around the axial direction relative to the distal cap, that the feedthrough arrangement is accommodated within a through-hole of the distal cap and that a surface structure of that at least one of the inner surface of the distal cap and the outer shell surface of the feedthrough provides one of a snap-fit and a press-fit locking connection with the respective other surface in the fixed state, wherein in the fixed state the surface structure forming the locking connection counteracts a movement of the distal cap and the feedthrough apart from each other in axial direction.

16. A manufacturing method for an implantable intracardiac device according to claim 14, comprising the following steps:
Providing the cylindrical housing with electric or electromagnetic components within the housing, the cylindrical feedthrough arrangement at the distal end of the housing, integrally formed with the housing or as separate element fixed at and hermetically sealed at the distal end face of the housing, and a pin-shaped electrode protruding from the distal end of the feedthrough arrangement and fixed within a recess of the feedthrough arrangement,
Providing the proximal cap, the distal cap and the base ring with the at least two tines,
Arranging the proximal cap, the base ring and the distal cap one above the other in axial direction such that the base ring is arranged between the proximal cap and the distal cap,
Fixing the proximal cap, the base ring and the distal cap to the feedthrough arrangement by application of an axial force onto a distal face of the distal cap and/or onto a proximal section of the feedthrough such that the base ring is fixed in an axial direction between the distal cap and the proximal cap and is rotatable around the axial direction relative to the distal cap, that the feedthrough is accommodated within a through-hole of the distal cap and that a surface structure of that at least one of the inner surface of the distal cap and the outer shell surface of the feedthrough provides one of a snap-fit and a press-fit locking connection with the respective other surface in the fixed state, wherein in the fixed state the surface structure forming the locking connection counteracts a movement of the distal cap and the feedthrough apart from each other in axial direction.
